(19) United States
(12) Patent Application Publication
(10) Pub. No.: US 2004/0091495 A1
(43) Pub. Date: May 13, 2004

(54) Protein

(76) Inventors: Lars Bjorck, Lund (SE); Anders Sjoholm, Lund (SE); Robert Janulczyk, Lund (SE); Gianni Pozzi, Souicille (IT); Francesco Iannelli, Andretta (IT)

Correspondence Address: Kalow & Springut, 488 Madison Avenue, New York, NY, 10022, US

(21) Appl. No.: 10/333,120

(22) PCT Filed: Jul. 19, 2001
(86) PCT No.: PCT/EP01/08409

(30) Foreign Application Priority Data
Jul. 20, 2000 (SE) 0002738.4

(51) Int. Cl. A61K 39/02; C12Q 1/68; C07H 21/04; C12P 21/02; C12N 1/21; C07K 14/315; C12N 15/74

(52) U.S. Cl. 424/185.1; 424/190.1; 435/6; 435/69.1; 435/252.3; 435/320.1; 530/350; 536/23.7

(57) ABSTRACT

The present invention provides proteins and peptides derived from Streptococcus pneuminiae, type 3, which all the complement factor H (fH). These proteins and peptides show a homology of at least 50%, preferably at least 60%, and most preferably at least 70% with the subsequence from Thr38 to Lys 149 of the sequence disclosed herein as SEQ ID NO: 1. The above subsequence, which is located in the amino-terminal of a surface protein isolated from Streptococcus Pneuminiae, type 3, is highly associated with binding of fH. In another aspect, the invention relates to proteins and peptides showing a homology of at least 85%, preferably at least 95%, and most preferably at least 99% with the Hic protein, the amino acid sequence of which is disclosed is SEQ ID NO: 1. The invention also provides vaccine compositions comprising the above proteins and peptides.

### FIELD OF THE INVENTION

The present invention relates to factor H-binding proteins and peptides, nucleic acid sequences encoding these proteins and peptides, antibodies specifically binding them and pharmaceutical compositions, especially vaccine compositions, containing them.

### BACKGROUND OF THE INVENTION

Despite the availability of effective antibiotics and polyvalent capsular polysaccharide vaccines, Streptococcus pneumoniae remains a significant cause of morbidity and mortality, causing conditions such as otitis media, community-acquired pneumoniae, septicemia and meningitis. Infants, the elderly and immunocompromised patients are particularly susceptible to pneumococcal infection.

The polysaccharide capsule of the pneumococcus has long been recognized as the major virulence determinant (1). Virulence varies with capsular serotype, but experiments with conversion of serotypes clearly demonstrate that other factors than the capsule play a significant role (2). A number of non-capsular virulence factors have also been extensively examined. Although their relative contribution to pneumococcal virulence remains unclear, it is apparent that proteins such as PspA, pneumolysin and PsaA play a role in pneumococcal virulence (3-5).

The classical and alternative pathways of complement are part of the innate immune system, and constitute an important line of defense against pneumococcal infection (6,7). There are many strategies by which bacteria can interfere with the function of the complement system (reviewed in (8)). For example, binding of the complement regulatory protein fH to bacterial surface proteins has been described by several groups (reviewed in (9)), although the precise consequences of such binding remain elusive. fH is a 150 kDa plasma protein composed of 20 short consensus repeats, and is the best characterised member of the factor H protein family (10). fH is a crucial protein in the regulation of complement. The critical step in the amplification loop of the alternative pathway is the formation of C3 convertase (C3bBb) from surface-deposited C3b and factor B. fH inhibits complement activation by preventing association of factor B with C3b, acting as a cofactor in C3b degradation by factor I, and promoting the dissociation of Bb from both C3 and C5 convertase.

Examples of bacterial surface structures interacting with fH include M and M-like proteins of S. pyogenes (11,12). Furthermore, YadA in Yersinia enterocolitica has been shown to inhibit complement activation by coating the bacterial surface with fH (13). Recently, two groups independently described inhibition of complement-mediated opsonophagocytosis in type 3 pneumococci. One study (14) suggested that PspA interferes with deposition of C3b and/or inhibits the alternative pathway C3 convertase. Another study (15) claimed that pneumococcal resistance to phagocytosis is mediated by hitherto unknown surface proteins binding fH. PspA does not contribute to this interaction, as a PspA-deficient mutant bound similar or even larger amounts of fH than the parent strain.

Vaccines against Streptococcus pneumoniae that are currently in use are all based upon capsule structures (carbohydrate antigens). Each serotype has a unique capsule structure. Therefore, vaccine compositions against Streptococcus pneumoniae contain numerous different capsular antigens in order to protect against the most common serotypes. Still, these compositions fail to provide an adequate protection in all individuals.

In particular some patients, such as those showing complement deficiency, are not sufficiently protected by these vaccine compositions. The protection against type 3 pneumococci is especially insufficient. Accordingly, there is a need for improved Streptococcus pneumoniae vaccine compositions.

### SUMMARY OF THE INVENTION

The present invention provides a polypeptide having the ability to bind factor H comprising: (a) the amino acid sequence of Thr38 to Lys149 of SEQ ID NO: 1 (b) a variant of (a) which is capable of binding factor H, or (c) a fragment of (a) or (b) of at least 20 amino acids in length which is capable of binding factor H, for use in prophylaxis or therapy.

The present invention also provides a vaccine composition comprising a polypeptide having an amino acid sequence selected from: (a) the amino acid sequence of Thr38 to Lys149 of SEQ ID NO: 1, (b) a variant of (a) which is capable of generating an immune response to Streptococcus pneumoniae or binding to an anti-protein Hic antibody, or (c) a fragment of (a) or (b) of at least 6 amino acids in length which is capable of generating an immune response against Streptococcus pneumoniae, or of binding to an anti protein Hic antibody.

In another aspect the invention provides a polypeptide of 15 to 800 amino acids in length comprising: (a) the amino acid sequence of SEQ ID NO: 1, or (b) a polypeptide showing sequence identity of at least 85%, preferably at least 95%, and most preferably at least 99% sequence identity with (a). Preferably the polypeptide has the ability to bind factor H.

The invention also provides a polynucleotide encoding a polypeptide, the polynculeotide comprising: (i) the nucleotide coding sequence of SEQ ID NO: 4 or a sequence complementary thereto, (ii) a nucleotide sequence which selectively hybridizes to said sequence (i) or a fragment thereof, or (iii) a nucleotide sequence which codes for a polypeptide having the same amino acid sequence as that encoded by a said sequence of (i) or (ii).

The present invention provides proteins and peptides derived from Streptococcus pneumoniae, type 3, which bind the complement factor H (fH). These proteins and peptides show a homology of at least 50%, preferably at least 60%, and most preferably at least 70% with the subsequence from Thr38 to Lys149 of the sequence disclosed herein as SEQ ID NO: 1. The above subsequence, which is located in the amino-terminal of a surface protein isolated from Streptococcus pneumoniae, type 3, is highly associated with binding of fH. In another aspect, the invention relates to proteins and peptides showing a homology of at least 85%, preferably at least 95%, and most preferably at least 99% with the Hic protein, the amino acid sequence of which is disclosed as SEQ ID NO: 1. The inventions also provides vaccine compositions comprising the above proteins and peptides.

### DESCRIPTION OF THE FIGURES

FIG. 1 discloses a comparison of Hic and PspC. Schematic representation of Hic and PspC. The signal peptide (SP) and the wall spanning region (W) are indicated;

FIG. 2 shows a sequence comparison of Hic and allelic variants of PspC, namely PspC6A (SEQ ID NO: 5), PspC2 (SEQ ID NO: 7), PspC19 (SEQ ID NO: 9), PspC19TIGR (SEQ ID NO: 10) and SpsA1 (SEQ ID NO: 11). ClustalW alignment of the NH2-terminal region from Hic (serotype 3) and allelic variants of PspC, including SpsA. Identical and similar residues are shaded in dark and light, respectively. Numbers in the protein names indicate the serotype of the strain from which the sequence was obtained. PspC.TIGR is from a serotype 4 strain. GenBank/EMBL accession numbers are as follows: PspC2, AF068645; PspC6A, AF068645; PspC19, AF068648; SpsA1, Y10818;

FIG. 3 relates to binding of radiolabelled factor H to a Hic-deficient mutant. Serial dilutions of PR218 (□) and the Hic-deficient mutant FP13 (♦) were incubated with radiolabelled fH. Binding is expressed as the percentage of added radioactivity. The data points are averages of three experiments with duplicate samples. Standard deviation is indicated by error bars;

FIG. 4 discloses binding of factor H to Hic. A competitive binding assay was performed by incubating PR218 bacteria (10⁹ CFU/ml) with radiolabelled fH in the presence of increasing concentrations of unlabelled fH (◯), GST (□), and GST:Hic(³⁹⁻²⁶¹) (♦);

FIG. 5 presents kinetic analysis of the interaction between Hic and factor H by surface plasmon resonance. One representative sensorgram (out of three) is shown. The concentration of the analyte (fH) was 2000, 667, 333, 167 and 83 nM, and the time scale has been adjusted so that 0 s represents the injection starting point.

FIG. 6 shows that Hic and factor H inhibit alternative pathway hemolysis. Rabbit erythrocytes were incubated with C2-deficient serum and a ldnetic study of hemolysis was performed. The influence of fH, GST, and/or GST:Hic³⁹⁻²⁶¹ on hemolysis was studied. Numbers represent hemolysis as a fraction of maximum hemolysis in the control (90-95%). Data shown are from one representative experiment (n=3). The curves represent control reaction (□), reaction with fH (♦) GST:Hic³⁹⁻²⁶¹ (□), GST (Δ), and a combination of fH and GST:Hic 39-261 (◯).

### DESCRIPTION OF THE SEQUENCES

SED ID NO: 1 is the amino acid sequence of protein Hic.

SED ID NOs: 2 and 3 are the primers used to amplify a portion of Hic.

SED ID NO: 4 is the DNA sequence encoding protein Hic.

SED ID NOs: 5 and 6 are the amino acid and encoding DNA sequence respectively for PspC6A.

SED ID NO: 7 and 8 are the amino acid and encoding DNA sequence respectively for PspC2.

SED ID NO: 9 is the amino acid sequence of PspC 19.

SED ID NO: 10 is the amino acid sequence of PspC 19 TIGR.

SED ID NO: 11 is the amino acid sequence of SpsA1.

### DETAILED DESCRIPTION OF THE INVENTION

General Terms and Introduction

The following abbreviations are used throughout the present application: fH, complement factor H; PspA, pneumococcal surface protein A; PsaA, pneumococcal surface antigen A; PspC, pneumococcal surface protein C; CbpA, choline-binding protein A; SpsA, Streptococcus pneumoniae secretory IgA binding protein; GST, glutathione S-transferase; PCR, polymerase chain reaction; SDS-PAGE, sodium dodecyl sulphate polyacrylamide gel electrophoresis.

As disclosed above, in a first aspect, the present application relates to proteins and peptides binding fH. These proteins and peptides are all comprise a derivative of the subsequence from thr38 to lys149 of amino-terminal of the Hic protein, whose amino acid sequence is enclosed herein as SEQ ID NO: 1. Peptides and proteins according to this first aspect show a homology of at least 50%, preferably at least 60%, and most preferably at least 70% with the subsequence from Thr38 to Lys149 of the sequence disclosed herein as SEQ ID NO: 1. Below, it will be shown that the amino-terminal part of the Hic protein is associated with binding fH. It will also be shown that the amino-terminal amino acid sequences shows substantial similarities with several other pneumococcal surface proteins, which probably also bind fH.

The polypeptides according to the first aspect of the invention have one or more of the following properties. Firstly, they may have the ability to bind to fH. As will be shown below, this feature makes them interesting as vaccine candidates against pneumococci. Secondly, peptides according to the invention may have the ability to stimulate a T-cell response. Thirdly, the peptides of the invention may stimulate a B-cell response.

In a second aspect, the present invention relates to peptides and proteins comprising at least 15 amino acids and which peptides and proteins show a homology of at least 85%, preferably at least 95%, and most preferably at least 99% with the amino acid sequence of the Hic protein disclosed as SEQ ID NO: 1.

The polypeptides according to the second aspect of the invention show a high homology to the Hic protein, which makes them interesting as vaccine candidates against pneumococci. Secondly, peptides according to this aspect of the invention may have the ability to stimulate a T-cell response. Thirdly, the peptides of this aspect of the invention may stimulate a B-cell response.

In an alternative aspect, the invention provides a polypeptide which comprises:

(a) the amino acid sequence of Thr38 to Lys149 of SEQ ID NO: 1,

(b) a variant of (a) which is capable of binding factor H, or

(c) a fragment of (a) or (b) of at least 20 amino acids in length which is capable of binding factor H.

In another aspect, the invention relates to a vaccine composition comprising a polypeptide which comprises:

(a) the amino acid sequence of Thr38 to Lys149 of SEQ ID NO: 1,

(b) a variant of (a) which is capable of generating an immune response to pneumococci, or binding to an anti-Hic antibody, or

(c) a fragment of (a) or (b) of at least 6 amino acids in length which is capable of generating an immune response against a pneumococcus, or of binding to an anti-Hic antibody.

The ability to bind factor H can be monitored by any suitable method. In particular, assays can be carried out using labelled factor H, for example, labelled with a radio label and monitoring binding to a peptide under investigation. Similarly, the ability of the peptides to bind anti-Hic antibodies can be monitored using any suitable assay, and in particular using antibodies which have been generated against full length protein Hic as shown in SEQ ID NO: 1 or a fragment thereof comprising the sequence of Thr38 to Lys149 of SEQ ID NO: 1. Similarly, the capability of the peptide to generate an immune response can be tested using a suitable animal model such as a murine model comprising administering the peptide under investigation to the animal, monitoring the generation of an immune response, either through the production of antibodies or through the production of T-cell responses such as cytotoxic T-cell responses. Preferred peptides which are able to generate a protective immune response can be identified by subjecting an animal so vaccinated with a lethal challenge of pneumococci. Similarly, the ability of the peptide to bind to anti-protein Hic antibodies can be assayed in vitro. An anti-protein Hic antibody can be generated by standard techniques using a protein of SEQ ID NO: 1 to generate a suitable antibody.

In a further aspect, the invention relates to a novel polynucleotide having a sequence which is:

(i) the nucleotide coding sequence of SEQ ID NO: 4 or a sequence complementary thereto,

(ii) a nucleotide sequence which selectively hybridizes to said sequence (i) or a fragment thereof, or

(iii) a nucleotide sequence which codes for a polypeptide having the same amino acid sequence as that encoded by a said sequence of (i) or (ii).

Polypeptides are provided which are encoded by a polynucleotide of the invention. The invention also relates to polynucleotides which encode a polypeptide of the invention. The invention also provides:

a recombinant vector comprising a polynucleotide of the invention, such as an expression vector in which the polynucleotide is operably linked to a regulatory sequence;

a host cell which is transformed for the polynucleotide of the invention;

a process of producing a polypeptide of the invention comprising maintaining a host cell transformed with a polynucleotide of the invention under conditions to provide expression of the polypeptide;

an antibody, monoclonal or polyclonal, specific for a polypeptide in accordance with the invention;

a method of vaccinating a patient against pneumococcal infection which method comprises administering to the patient an effective amount of a polypeptide or polynucleotide according to the invention.

In another aspect, the invention relates to an assay to identify an agent which inhibits the binding of factor H to protein Hic. The assay may comprise incubating a polypeptide of the invention having the ability to bind factor H with factor H in the presence of the substance under test and monitoring for binding of the polypeptide of the invention to factor H. Methods for monitoring protein interactions are well known in the art. The polypeptide of the invention may be provided in isolated form. In the alternative, a bacterium expressing a polypeptide of the invention may be provided and incubated with factor H. Substances which are identified which inhibit the binding of factor H to a polypeptide of the invention may be used as an antibiotic or in the treatment of an individual suffering from pneumococcal infection.

The invention also provides formulating an agent identified as an inhibitor of the binding of factor H to a polypeptide of the invention with a pharmaceutically acceptable carrier.

Accordingly, the present invention provides a method for the treatment of a Streptococcus pneumoniae infection, which method comprises identifying a substance which inhibits binding of factor H to a polypeptide of the invention and administering a therapeutically effective amount of the substance to a patient in need thereof. A therapeutically effective amount of the substance is an amount which alleviates the symptoms of the Streptococcus pneumoniae infection or otherwise improves the condition of the patient suffering from a Streptococcus pneumoniae infection. Preferably the substance administered to a patient is formulated with a pharmaceutically acceptable carrier.

A DNA sequence encoding the Hic protein is disclosed as SEQ ID NO: 4. The nucleic acid sequences of the present invention are preferably DNA, though they may be RNA. It will be obvious to those of skill in the art that, in RNA sequences according to the invention, the T residues will be replaced by U. Nucleic acid sequences of the invention will typically be in isolated or substantially isolated form. For example up to 80, up to 90, up to 95 or up to 100% of the nucleic acid material in a preparation of a nucleic acid of the invention will typically be nucleic acid according to the invention. Alterations, isolations or syntheses of nucleic acid sequences according to the invention may be performed by any conventional method, for example by the methods of Sambrook et al (Molecular Cloning: A Laboratory Manual; 1989).

For example, the sequences of the invention include sequences that are capable of selective hybridisation to those encoding the proteins or peptides of the invention or the complementary strands thereof and that encode a polypeptide having one or more of the properties defined above.

Such hybridisation may be carried out under any suitable conditions known in the art (see Sambrook et al (1989): Molecular Cloning: A Laboratory Manual). For example, if high stringency is required, suitable conditions include 0.2×SSC at 60° C. If lower stringency is required, suitable conditions include 2×SSC at 60° C.

Preferably, a polynucleotide of the invention can hybridize to SEQ ID NO: 4, or its complement at a level significantly above background. Background hybridization may occur, for example, because of other cDNAs present in a cDNA library. The signal level generated by the interaction between a polynucleotide of the invention and the sequence of SEQ ID NO: 4 is typically at least 10 fold, preferably at least 100 fold, as intense as interactions between other polynucleotides and the sequence of SEQ ID NO: 4. The intensity of interaction may be measured, for example, by radiolabelling the probe, e.g. with ³²P. Selective hybridization is typically achieved using conditions of medium to high stringency (for example 0.1 to 0.2×SSC at from about 50° C. to about 60° C.).

A nucleotide sequence capable of selectively hybridizing to the DNA sequence of SEQ ID NO: 4 or to the sequence complementary to that coding sequence will be generally at least 80%, preferably at least 90% and more preferably at least 95%, homologous to the sequence of SEQ ID NO: 4 or its complement over a region of at least 20, preferably at least 30, for instance at least 40, 60 or 100 or more contiguous nucleotides or, indeed, over the full length of the coding sequence. Thus there may be at least 85%, at least 90% or at least 95% nucleotide identity over such regions.

Any combination of the above mentioned degrees of homology and minimum size may be used to define polynucleotides of the invention, with the more stringent combinations (i.e. higher homology over longer lengths) being preferred. Thus for example a polynucleotide which is at least 85% homologous over 25, preferably over 30, nucleotides forms one aspect of the invention, as does a polynucleotide which is at least 90% homologous over 40 nucleotides.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSP's containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Also included within the scope of the invention are sequences that differ from those defined above because of the degeneracy of the genetic code and encode the same polypeptide having one or more of the properties defined above.

Nucleic acid sequences of the invention will preferably be at least 30 bases in length, for example up to 50, up to 100, up to 200, up to 300, up to 400, up to 500, up to 600, up to 800, up to 1000 bases, up to 2000 bases or up to 3000 bases.

Nucleic acid sequences of the invention may be extended at either or both of the 5′ and 3′ ends. Such extensions may be of any length. For example, an extension may comprise up to 10, up to 20, up to 50, up to 100, up to 200 or up to 500 or more nucleic acids. Thus, the nucleic acid sequences of the invention may be extended at either or both of the 5′ and 3′ ends by any non-wild-type sequence.

The polypeptides of the invention are derived from the amino acid sequence shown as SEQ ID NO: 1. Thus, the polypeptides of the invention are not limited to the polypeptide of SEQ ID NO: 1. Rather, the polypeptides of the invention also include polypeptides with sequences closely related to that of SEQ ID NO: 1 that are able to bind fH. Alternatively, the polypeptides of the invention comprises very similar or identical antigenic determinants compared to the Hic protein despite the fact that they do not bind to fH. All these sequences may be prepared by altering that of SEQ ID NO: 1 by any conventional method, or isolated from any organism or made synthetically. Such alterations, isolations or syntheses may be performed by any conventional method, for example by the methods of Sambrook et al (Molecular Cloning: A Laboratory Manual; 1989). In particular, polypeptides related to that of SEQ ID NO: 1 may be prepared by modifying a DNA sequence encoding the Hic protein expressing them recombinantly.

The polypeptides of the invention may accordingly have less than 100% sequence identity with that of SEQ ID NO: 1. Thus, polypeptides of the invention may include substitutions, deletions, or insertions that distinguish them from SEQ ID NO: 1 as long as these do not destroy the ability to bind fH, or as long as they show similar antigenic properties as the Hic protein.

A substitution, deletion or insertion may suitably involve one or more amino acids, typically from one to five, one to ten or one to twenty amino acids, for example, a substitution, deletion or insertion of one, two, three, four, five, eight, ten, fifteen, or twenty amino acids. Typically, a fH-binding polypeptide of the first aspect of the invention has at least 50%, at least 60%, or at least 70% sequence identity to the subsequence from thr38 to lys149 of SEQ ID NO: 1. Preferably the polypeptide has at least 80%, 90%, 95%, 97% or 99% identity with SEQ ID NO: 1. Typically, a polypeptide of the second aspect of the invention has at least 85%, at least 95, or at least 99% sequence identity to a subsequence that can be derived from all parts of the sequence of SEQ ID NO: 1, and preferably to a sequence of 15 to 600 contiguous amino acids of SEQ ID NO: 1.

In general, the physicochemical nature of the sequence of SEQ ID NO: 1 should be preserved in a polypeptide of the invention. Such sequences will generally be similar in charge, hydrophobicity and size to that of SEQ ID NO: 1. Examples of substitutions that do not greatly affect the physicochemical nature of amino acid sequences are those in which an amino acid from one of the following groups is substituted by a different amino acid from the same group:

H, R and K

I, L, V and M

A, G, S and T

D, E, Q and N.

Where polypeptides of the invention are synthesised chemically, D-amino acids (which do not occur in nature) may be incorporated into the amino acid sequence at sites where they do not affect the polypeptides biological properties. This reduces the polypeptides' susceptibility to proteolysis by the recipient's proteases.

The nucleic acid sequences encoding the polypeptides of the invention may be extended at one or both ends by any non-wild-type sequence.

Thus, the polypeptides of the invention may be extended at the C-terminal by an amino acid sequence of any length. For example, an extension may comprise up to 5, up to 10, up to 20, up to 50, or up to 100 or 200 or more amino acids. A C-terminal extension may have any sequence apart from that which is C-terminal to the sequence of the invention (or the native sequence from which it is derived) in native Hic protein. Thus, the polypeptides of the invention may be extended at the C-terminal by any non-wild-type sequence.

The polypeptides of the invention may be attached to other polypeptides, proteins or carbohydrates that enhance their antigenic properties. Thus, polypeptides of the invention may be attached to one or more other antigenic polypeptides. These additional antigenic polypeptides may be derived from S. pneumoniae or from another organism. Possible additional antigenic polypeptides include heterologous T-cell epitopes derived from other S. pneumoniae proteins or from species other than S. pneumoniae. Heterologous B-cell epitopes may also be used. Such heterologous T-cell and or B-cell epitopes may be of any length and epitopes of up to 5, up to 10 or up to 20 amino acids in length are particularly preferred. Possible carbohydrates that can be added are capsular antigens. These additional antigenic polypeptides may be attached to the polypeptides of the invention chemically. Alternatively, one or more additional antigenic sequences or carbohydrate groups may comprise an extension to a polypeptide of the invention.

A polypeptide of the invention may be subjected to one or more chemical modifications, such as glycosylation, sulphation, COOH-amidation or acylation. In particular, polypeptides that are acetylated at the N-terminus are preferred, as are polypeptides having C-terminal amide groups. Preferred polypeptides may have one or more of these modifications. For example, particularly preferred peptides may have a C-terminal amide group and N-terminal acetylation.

A polypeptide of the invention may form part of a larger polypeptide comprising multiple copies of the sequence of one or more of SEQ ID NO: 1 or a sequences related to them in any of the ways defined herein.

Polypeptides of the invention typically comprise at least 15 amino acids, for example 15 to 20, 20 to 50, 50 to 100 or 100 to 200 or 200 to 300 or 300 to 400 or 400 to 500 or 500 to 600 or 600 to 700 or 700 to 800 amino acids.

Polypeptides according to the invention may be purified or substantially purified. Such a polypeptide in substantially purified form will generally form part of a preparation in which more than 90%, for example up to 95%, up to 98% or up to 99% of the peptide material in the preparation is that of a polypeptide or polypeptides according to the invention.

The nucleic acid sequences and polypeptides of the invention were originally derived from S. pneumoniae. However, nucleic acid sequences and/or polypeptides of the invention may also be obtained from other organisms, typically bacteria, especially other streptococci. They may be obtained either by conventional cloning techniques or by probing genomic or cDNA libraries with nucleic acid sequences according to the invention. This can be done by any conventional method, such as the methods of Sambrook et al (Molecular Cloning: A Laboratory Manual; 1989).

A nucleic acid sequence according to the invention may be included within a vector, suitably a replicable vector, for instance a replicable expression vector.

A replicable expression vector comprises an origin of replication so that the vector can be replicated in a host cell such as a bacterial host cell. A suitable vector will also typically comprise the following elements, usually in a 5′ to 3′ arrangement: a promoter for directing expression of the nucleic acid sequence and optionally a regulator of the promoter, a translational start codon and a nucleic acid sequence according to the invention encoding a polypeptide having one or more of the biological properties of Hic protein. A non-replicable vector lacks a suitable origin of replication whilst a non-expression vector lacks an effective promoter.

The vector may also contain one or more selectable marker genes, for example an ampicillin resistance gene for the identification of bacterial transformants. Another possible marker gene is the kanamycin resistance gene. Optionally, the vector may also comprise an enhancer for the promoter. If it is desired to express the nucleic acid sequence of the invention in a eucaryotic cell, the vector may also comprise a polyadenylation signal operably linked 3′ to the nucleic acid encoding the functional protein. The vector may also comprise a transcriptional terminator 3′ to the sequence encoding the polypeptide of the invention.

The vector may also comprise one or more non-coding sequences 3′ to the sequence encoding the polypeptide of the invention. These may be from S. pneumoniae (the organism from which the sequences of the invention are derived) or the host organism, which is to be transformed with the vector or from another organism.

In an expression vector, the nucleic acid sequence of the invention is operably linked to a promoter capable of expressing the sequence. “Operably linked” refers to a juxtaposition wherein the promoter and the nucleic acid sequence encoding the polypeptide of the invention are in a relationship permitting the coding sequence to be expressed under the control of the promoter. Thus, there may be elements such as 5′ non-coding sequence between the promoter and coding sequence. These elements may be native either to S. pneumoniae or to the organism from which the promoter sequence is derived or to neither organism. Such sequences can be included in the vector if they enhance or do not impair the correct control of the coding sequence by the promoter.

The vector may be of any type. The vector may be in linear or circular form. For example, the vector may be a plasmid vector. Those of skill in the art will be able to prepare suitable vectors comprising nucleic acid sequences encoding polypeptides of the invention starting with widely available vectors which will be modified by genetic engineering techniques such as those described by Sambrook et al (Molecular Cloning: A Laboratory Manual; 1989). Preferred starting vectors include plasmids that confer kanamycin resistance and direct expression of the polypeptide of the invention via a tac promoter.

In an expression vector, any promoter capable of directing expression of a sequence of the invention in a host cell may be, operably linked to the nucleic acid sequence of the invention. Suitable promoters include the tac promoter.

Such vectors may be used to transfect or transform a host cell. Depending on the type of vector, they may be used as cloning vectors to amplify DNA sequences according to the invention or to express this DNA in a host cell.

A further embodiment of the invention provides host cells harbouring vectors of the invention, i.e. cells transformed or transfected with vectors for the replication and/or expression of nucleic acid sequences according to the invention. The cells will be chosen to be compatible with the vector and may for example be bacterial cells.

Transformed or transfected bacterial cells, for example E. coli cells, will be particularly useful for amplifying nucleic acid sequences of the invention as well as for expressing them as polypeptides.

The cells may be transformed or transfected by any suitable method, such as the methods described by Sambrook et al (Molecular cloning: A Laboratory Manual; 1989). For example, vectors comprising nucleic acid sequences according to the invention may be packaged into infectious viral particles, such as retroviral particles. The constructs may also be introduced, for example, by electroporation, calcium phosphate precipitation, and biolistic methods or by contacting naked nucleic acid vectors with the cells in solution.

In the said nucleic acid vectors with which the host cells are transformed or transfected, the nucleic may be DNA or RNA, preferably DNA.

The vectors with which the host cells are transformed or transfected may be of any suitable type. The vectors may be able to effect integration of nucleic acid sequences of the invention into the host cell genome or they may remain free in the cytoplasm. For example, the vector used for transformation may be an expression vector as defined herein.

The present invention also provides a process of producing polypeptides according to the invention. Such a process will typically comprise transforming or transfecting host cells with vectors comprising nucleic acid sequences according to the invention and expressing the nucleic acid sequence in these cells. In this case, the nucleic acid sequence will be operably linked to a promoter capable of directing its expression in the host cell. Desirably, such a promoter will be a “strong” promoter capable of achieving high levels of expression in the host cell. It may be desirable to overexpress the polypeptide according to the invention in the host cell. Suitable host cells for this purpose include yeast cells and bacterial cells, for example E. coli cells, a particularly preferred E. coli strain being E. coli K12 strain BL 21. However, other expression systems can also be used, for example baculovirus systems in which the vector is a baculovirus having in its genome nucleic acid encoding a polypeptide of the invention and expression occurs when the baculovirus is allowed to infect insect cells.

The thus produced polypeptide of the invention may he recovered by any suitable method known in the art. Optionally, the thus recovered polypeptide may be purified by any suitable method, for example a method according to Sambrook et al (Molecular Cloning: A Laboratory Manual).

The polypeptides of the invention may also be synthesised chemically using standard techniques of peptide synthesis. For shorter polypeptides, chemical synthesis may be preferable to recombinant expression. In particular, peptides of up to 20 or up to 40 amino acid residues in length may desirably be synthesised chemically.

The nucleic acid sequences of the invention may be used to prepare probes and primers. These will be useful, for example, in the isolation of genes having sequences similar to that of SEQ ID NO: 4. Such probes and primers may be of any suitable length, desirably from 10 to 100, for example from 10 to 20, 20 to 50 or 50 to 100 bases in length. Examples of such probes are disclosed herein as SEQ ID NO: 2 and SEQ ID NO: 3.

Polynucleotides or primers of the invention may carry a revealing label. Suitable labels include radioisotopes such as ³²P or ³⁵S, enzyme labels or other protein labels such as biotin. Such labels may be added to polynucleotides or primers of the invention and may be detected using techniques known per se.

Polynucleotides or primers of the invention or fragments thereof, labelled or uLnlabelled, may be used by a person skilled in the art in nucleic acid-based tests for detecting or sequencing protein Hic in a sample.

Such tests for detecting generally comprise bringing a sample containing DNA or RNA into contact with a probe comprising a polynucleotide or primer of the invention under hybridizing conditions and detecting any duplex formed between the probe and nucleic acid in the sample. Such detection may be achieved using techniques such as PCR or by immobilizing the probe on a solid support, removing nucleic acid in the sample which is not hybridized to the probe, and then detecting nucleic acid which has hybridized to the probe. Alternatively, the sample nucleic acid may be immobilized on a solid support, and the amount of probe bound to such a support can be detected.

The probes of the invention may conveniently be packaged in the form of a test kit in a suitable container. In such kits the probe may be bound to a solid support where the assay formats for which the kit is designed requires such binding. The kit may also contain suitable reagents for treating the sample to be probed, hybridizing the probe to nucleic acid in the sample, control reagents, instructions, and the like.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described in connection with expression vectors.

The present invention also provides antibodies to the polypeptides of the invention. These antibodies may be monoclonal or polyclonal. For the purposes of this invention, the term “antibody”, includes fragments of whole antibodies which retain their binding activity for a target antigen. Such fragments include Fv, F(ab′) and F(ab′)₂ fragments, as well as single chain antibodies.

The antibodies may be produced by any method known in the art, such as the methods of Sambrook et al (Molecular Cloning: A Laboratory Manual; 1989). For example, they may be prepared by conventional hybridoma techniques or, in the case of modified antibodies or fragments, by recombinant DNA technology, for example by the expression in a suitable host vector of a DNA construct encoding the modified antibody or fragment operably linked to a promoter. Suitable host cells include bacterial (for example E coli), yeast, insect and mammalian cells. Polyclonal antibodies may also be prepared by conventional means which comprise inoculating a host animal, for example a rat or a rabbit, with a peptide of the invention and recovering immune serum.

The present invention also provides pharmaceutical compositions comprising polypeptides of the invention. Compositions comprising polypeptides of the invention that include T-cell and/or B-cell epitopes may be used as vaccines against infections caused by pneumococci.

A range of mammalian species can be vaccinated against infections caused by type 3 streptococci using the polypeptides of the invention. Vaccination of humans is particularly desirable.

The compositions of the invention may be administered to mammals including humans by any route appropriate. Suitable routes include topical application in the mouth, oral delivery by means of tablets or capsule and parenteral delivery, including subcutaneous, intramuscular, intravenous and intradermal delivery. Preferred routes of administration are injection, typically subcutaneous or intramuscular injection, with a view to effecting systemic immunisation.

As previously indicated, polypeptides according to the invention may also be mixed with other antigens of different immunogenicity.

The compositions of the invention may be administered to the subject alone or in a liposome or associated with other delivery molecules. The effective dosage depends on many factors, such as whether a delivery molecule is used, the route of delivery and the size of the mammal being vaccinated. Typical doses are from 0.1 to 100 mg of the polypeptide of the invention per dose, for example 0.1 to 1 mg, and 1 to 5 mg, 5 to 10 mg and 10 to 100 mg per dose. Doses of from 1 to 5 mg are preferred.

Dosage schedules will vary according to, for example, the route of administration, the species of the recipient and the condition of the recipient. However, single doses and multiple doses spread over periods of days, weeks or months are envisaged. A regime for administering a vaccine composition of the invention to young human patients will conveniently be: 6 months, 2 years, 5 years and 10 years, with the initial dose being accompanied by adjuvant and the subsequent doses being about ½ to ¼ the level of polypeptide in the initial dose. The frequency of administration can, however, be determined by monitoring the antibody levels in the patient.

While it is possible for polypeptides of the invention to be administered alone it is preferable to present them as pharmaceutical formulations. The formulations of the present invention comprise at least one active ingredient, a polypeptide of the invention, together with one or more acceptable carriers thereof and optionally other therapeutic ingredients. The carrier or carriers must be “acceptable” in the sense of being compatible with the other ingredients of the formulation and not deleterious to the recipients thereof, for example, liposomes.

The preparation of vaccines which contain an immunogenic polypeptide(s) as active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Examples of adjuvants which may be effective include but are not limited to: aluminium hydroxide, N-acetyl-muramyl-L-threonyl-D-isoglutamin (thr-MDP), N-acetyl-nor-muramyl-L-alanyl-D-isoglutamine (CGP 11637, referred to as nor-MDP), N-acetylmuramyl-L-alanyl-D-isoglutamnyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against an immunogenic polypeptide containing Hic antigenic sequence resulting from administration of this polypeptide in vaccines which are also comprised of the various adjuvants.

The vaccines are conventionally administered parentally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccarine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The polypeptides of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Formulations suitable for parenteral administration include aqueous and non-aqueous sterile injection solutions which may contain anti-oxidants, buffers, bacteriostatis, bactericidal antibiotics and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs.

Of the possible formulations, sterile pyrogen-free aqueous and non-aqueous solutions are preferred. Also preferred are formulations in which the polypeptides of the invention are contained in liposomes. Injection solutions and suspensions may be prepared extemporaneously from sterile powders, granules and tablets of the kind previously described.

Oral methods of administration may produce a systemic effect. Orally active preparations can be formulated in any suitable carrier, such as a gel, toothpaste, mouthwash or chewing gum.

It should be understood that in addition to the ingredients particularly mentioned above the formulations of this invention may include other agents conventional in the art having regard to the type of formulation in question.

Accordingly, the present invention provides a method of vaccinating a mammalian host against infections caused by pneumococci or treating such infections, which method comprises administering to the host an effective amount of a pharmaceutical composition as described above, for example a vaccine composition.

Antibodies, including monoclonal antibodies, and fragments thereof such as Fab fragments can be formulated for passive immunisation as indicated above for the formulation of including polypeptides of the invention. Preferred formulations for passive immunisation include solid or liquid formulations such as gels, toothpastes, mouth-washes or chewing gum.

A further aspect of the present invention is a naked nucleic acid vaccine. In this embodiment, the vaccine composition comprises a nucleic acid, typically an isolated nucleic acid, preferably DNA, rather than a polypeptide. The nucleic acid is injected in to a mammalian host and expressed in vivo, generating a polypeptide of the invention. This stimulates a T-cell response, which leads to protective immunity against Streptococcus pneumoniae, for example against dental caries, in the same way as direct vaccination with a polypeptide of the invention.

Nucleic acid vaccination can be carried out with any nucleic acid according to the invention as long as it encodes a polypeptide that stimulates a T-cell and or B-cell response. These nucleic acids will typically be included within an expression vectors as defined above. In such an expression vector, the nucleic acid according to the invention will typically be operably linked to a promoter capable of directing its expression in a mammalian host cell. For example, promoters from viral genes that are expressed in the mammalian cells such as the cytomegalovirus (CMV) immediate early gene promoter are suitable. Also suitable are promoters from mammalian genes that are expressed in many or all mammalian cell types such as the promoters of “housekeeping” genes. One such promoter is the p-hydroxymethyl-CoA-reductase(HMG) promoter (Gautier et al (1989): Nucleic Acids Research; 17,8839).

For naked nucleic acid vaccination, it is preferred that the nucleic acid sequence according to the invention is incorporated into a plasmid vector, since it has been found that covalent closed circle (CCC) plasmid DNA can be taken up directly by muscle cells and expressed without being integrated into the cells' genomic DNA (Ascadi et al (1991): The New Biologist; 3, 71-81). Naked nucleic acid vaccine may be prepared as any of the types of formulation mentioned above in respect of conventional polypeptide-based vaccines. However, formulations suitable for parenteral injection, especially intramuscular injection, are preferred. Naked nucleic acid vaccines may be delivered in any of the ways mentioned above in respect of conventional polypeptide-based vaccines but intramuscular injection is preferred.

Alternatively, nucleic acid vaccines may be provided suitable for administration by particle bombardment, or in formulations comprising liposomes or cationic lipid formulation such as lipofectin or other suitable carriers.

Accordingly, the present invention provides a vaccine composition comprising a nucleic acid sequence or vector as described above and an acceptable carrier.

Introduction to the Experimental Work

Complement-dependent opsonophagocytosis is a crucial defense against infection with Streptococcus pneumoniae (33). Two recent studies discuss how type 3 pneumococci may subvert the normal function of the complement system. One study (14) showed that the alternative pathway of complement is essential for efficient clearance of bacteria, and that pneumococcal interference with complement activation is a virulence determinant. Based on comparisons of PspA-negative mutants with wild type bacteria the researchers claim that PspA blocks recruitment of the alternative pathway, by an unknown mechanism. Another study (15) describes the binding of complement factor H to trypsin-sensitive structures at the surface of pneumococci, independent of previous activation of complement. Notably, the study rules out any major contribution of PspA with respect to binding of fH. The findings indicate that factor H binding proteins might constitute an independent virulence factor.

The present investigation describes a novel pneumococcal surface protein, responsible for the binding of fH in type 3 pneumococci. The gene encoding the protein (Hic) is found in the locus of pspC, a possible virulence determinant and protective antigen, but shows highly a typical characteristics compared to previously described pspC alleles. In a study where a number of pspC alleles were sequenced (16) the type 3 strain was considered pspC-negative, since there was no protein reacting with PspC-antibodies and PCR experiments failed to amplify the pspC gene. Apart from a region in the NH2-terminal part of the protein, there is little sequence homology between Hic and PspC. Furthermore, Hic is anchored to the cell wall by the presence of an LPXTGX motif while PspC harbors the choline binding motif. In this respect, Hic is similar to the M protein in S. pyogenes, also known to bind fH (11). A Hic-deficient mutant failed to absorb fH from human plasma, in contrast to the wild type strain. This mutant did not bind radiolabelled fH; indicating that most or all of the fH binding to PR28 bacteria is due to the presence of Hic at the pneumococcal surface. Although an extensive screening was not performed, most of the strains examined showed binding of fH, suggesting that this phenotype is not confined to type 3 strains. Furthermore, the recombinant fH-binding fragment of Hic could compete with binding of radiolabelled fH to pneumococci of different serotypes. PspC may confer fH binding to pneumococci by virtue of the region similar to Hic, but it cannot be excluded that other surface structures interact with the same part of fH as Hic does. Surface plasmon resonance experiments with the fusion between GST and the NH2-terminal Hic region (GST:Hic³⁹⁻²⁶¹) and fH showed that the interaction has high affinity.

The presence of a similarity region in the NH2-terminal part of various PspC proteins and Hic, offers the interesting possibility that this rather variable region, contained in the recombinantly expressed Hic fragment, is responsible for factor H binding in many pneumococcal strains. It has previously been shown that PspC is a protective antigen, and the NH2-terminal region probably has to undergo significant genetic variation in order to avoid eliciting specific antibodies. This, however, does not preclude specific binding of fH. In comparison, the M5 and M6 proteins of S. pyogenes have been shown to bind factor H-like protein 1 by their hypervariable region (12). Similarly, several M-like proteins bind another complement regulatory protein, C4 binding protein (34), by the hypervariable NH2-terminal region. We found that a type 2 mutant strain that expressed an NH2-terminally truncated form of PspC failed to absorb fH from plasma, unlike the parent strain D39. Although not conclusive, since the truncation also involved a part of PspC that shows no homology with Hic, this experiment supports the idea that fH binding could be 15′ mediated by the NH2-terminal regions of both PspC and Hic.

Many studies have showed that interference with the function of the complement system is a highly relevant aspect of pneumococcal virulence. More specifically, the binding of fH has been shown to correlate with resistance to opsonophagocytosis. A previous study showed that type 3 pneumococci, despite C3b deposition on both cell wall and capsule, strongly resist phagocytosis (29). Our data show that hic, a highly a typical pspC allele, encodes the major fH-binding protein of type 3 pneumococci. The complement inhibitory function of fH is unaffected by the interaction with Hic. By accumulating an active complement inhibitor at the pneumococcal surface, Hic may act alone or in concert with PspA to block deposition of C3b and concomitant opsonophagocytosis. As previously described, a putative C3 proteinase in types 3, 4 and 14 pneumococci (35) should have similar effects. The present observation that a region of Hic shows an intrinsic capacity to inhibit the alternative pathway further underlines the significance of this virulence mechanism. In conclusion, pneumococci appear highly prone to interfere with the complement system. Binding of fH, by different mechanisms, has also been described for group A streptococci (11), Y. enterocolitica (13), and N. gonorrhoeae (36,37) and may represent a widespread theme in bacterial adaptation to the human host.

The present invention will now be described with reference to the enclosed figures, in which:

Experimental Procedures

Bacterial Strains

Strains of S. pneumoniae used in this study are described in Table 1. In unencapsulated strains PR201, PR212, PR215, and PR218 the whole capsule locus is deleted and substituted with a kanamycin (Km) resistance cassette (provided by F. Ianelli, B. J. Pearce and G. Pozzi). Pneumococci were grown at 37° C. in TSB (Difco) or on TSA (TSB with agar) supplemented with 3% horse blood. Where appropriate, kanamycin (500 μg/ml) or chloramphenicol (3 μg/ml) was added. Bacteria used for binding assays were grown in Todd-Hewitt broth (Difco), supplemented with 0.2% Yeast extract (Difco). Escherichia coli strain DH5α was grown in Luria-Broth (Difco) or on LB-agar, supplemented with ampicillin (50 μg/ml) when containing pGEX.

DNA Methods, Cloning, and Sequencing

By PCR SOEing (19), a chloramphenicol transferase cassette was flanked by sequences found up and downstream of hic. PR218 was transformed with this construct. By double cross-over mutagenesis the hic gene was consequently replaced with the cat cassette, generating the hic-deficient mutant FP 13.

Oligonucleotides HICfl (5′-TGGGATCCCAGAGAAGGAGGTAAC TAC-3′, SEQ ID NO: 2) and HICr1 (5′-GGAGCCTGAATTCGACGAAG-3′, SEQ ID NO: 3), containing BamHI and EcoRI restriction sites respectively, were used in a polymerase chain reaction (PCR) to amplify DNA corresponding to amino acids 39 to 261 in Hic. The PCR was performed with Taq polymerase (Gibco BRL), and consisted of 30 cycles at 94° C. for 1 min., 50° C. for 1 min., and 72° C. for 1 min., followed by a final extension at 72° C. for 7 min. Template was prepared by resuspending bacterial colonies in water, boiling for 5 min., and removal of bacterial debris by centrifuging at 13000×g. The PCR-amplified fragment was gel-purified with Sephaglas Bandprep (Pharmacia Biotech), digested with BamHI and EcoRI (Pharmacia Biotech) and ligated with likewise digested vector pGEX-5×-3 (Pharmacia Biotech) using T4 DNA ligase (Pharmacia Biotech). Plasmid pGEX-5×-3:hic(39-261) was then electroporated into DH5α E. coli according to the GST gene fusion system protocol (Pharmacia Biotech). Transformants were screened for presence of insert by plasmid mini-preps and restriction enzyme digestion. The clone used for overexpression of the fusion protein GST:Hic³⁹⁻²⁶¹ was verified by purifying the plasmid and sequencing the complete insert. Fusion protein was affinity purified according to the instructions in the GST gene fusion system manual (Pharmacia Biotech).

Ligand Binding and Protein Methods

Plasma absorption experiments were performed with log-phase pneumococci (OD₆₀₀ appr. 0.4). Bacteria were washed twice in PBS, pH 7.4, containing 0.05% Tween 20 (PBST), and the bacterial concentration was adjusted to 2×10¹⁰ cells/ml. 100 μl of bacteria were incubated for 1 h with 100 μl of human plasma. Bacteria were washed five times with PB ST, and bound proteins were eluted from the cells with 100 μl 0.1 M glycine/HCl, pH 2.0. The pH of the eluted material was adjusted to appr. 7 with 1 M Tris. C3-deficient serum was obtained from a patient with selective complete C3 deficiency (C3<1 mg/liter), kindly provided by Dr. G. Eggertsen, Huddinge Hospital, Sweden.

Protein samples were separated by SDS-PAGE (20) containing 8-12% acrylamide. Proteins were blotted onto an Immobilon-P™ PVDF-membrane (Millipore) as described (21). Rabbit polyclonal antiserum against fH diluted 1: 1000 was the source of primary antibodies. Horseradish peroxidase-conjugated anti-rabbit goat antibodies (Bio-Rad, Bio-Rad Laboratories, CA) were used as secondary antibodies, and detection of immuno-reactive bands was performed by chemiluminescence as described (22).

Slot blot, Western blot and bacterial binding assays were performed with f (Sigma), radiolabelled with ¹²⁵I using the Iodobeads kit (Pierce). Unincorporated ¹²⁵I was removed by gel filtration on Sephadex G-25 (Pharmacia Biotech). Slot blots were performed by applying 5, 1, 0.02 and 0.004 μg of purified protein onto nitrocellulose membranes in a slot blot apparatus (Schleicher & Schuell). The membrane was blocked for 4×20 min. in PBST containing 0.25% (w/v) gelatin (Difco). Radiolabelled fH (200000 cpm/ml) was then added, and the membrane was incubated for 1 h at room temperature. The membrane was washed for 4×20 min. in PBST, 0.5 M NaCl, and membrane-associated radioactivity was visualized by exposure onto a phosphoimaging plate (Fuji Photo Film). Western blot membranes were treated in the same way. Binding assays with pneurnococci were performed as described (23), using log-phase bacteria (OD₆₀₀ appr. 0.4).

Surface plasmon resonance was performed by coupling affinity-purified anti-GST antibodies (Biacore) onto sensor chip CM5 (Biacore) by standard amine coupling, according to the manufacturers instructions. Appr. 10000 resonance units (RU) of antibodies were coupled. Each cycle of analysis was then commenced by immobilization of 500-1500 RU of GST or GST:Hic³⁹⁻²⁶¹ (10 μg/ml), followed by injection of fH (2-0.127 μM). The kinetic studies were performed in PBST. Each cycle was terminated by the regeneration of the chip with 10 mM glycine pH 2.2. Global analysis of data was performed with multiple models in BiaEvaluation 3.0.

Hemolysis Assay

A previously described assay for the measurement of fH-mediated inhibition of the alternative pathway was employed (24). Rabbit erythrocytes (National Institute of Veterinary Medicine, Uppsala, Sweden) were washed and suspended at 5×10⁸ cells/ml. The serum of a patient with homozygous C2-deficiency (25) was used as a source of cbmplement. Highly purified fH was also kindly provided by Dr. L. Truedsson. The hemolytic reaction was performed in veronal-buffered saline with 16 mM Mg²⁺ mM EGTA, 4 and 0.11% gelatine. The concentration of C2-deficient serum producing about 80% hemolysis in the assay system was determined in preliminary experiments. Final incubation mixtures contained C2-deficient serum diluted 1/12 (200 μl) with or without additional proteins (fH, GST, or GST:Hic³⁹⁻²⁶¹, at 30, 90 or 60 μM, respectively) and 2.5×10⁷ rabbit erythrocytes (50 μl). The erythrocytes were added 5 min after the other reagents. After 5-40 min reactions were stopped by the addition of 750 μl cold VBS, 10 mM EDTA. Samples were centrifuged at 3000 rpm for 5 min., and the supernatants were removed for measurement of light absorbance at 412 nm.

Bioinformatics

Sequence comparisons were performed with MacVector 6.5.3 (Oxford Molecular, Oxford, United Kingdom). Database searches utilized the Entrez server at The National Institute for Biotechnology Information. The sequence of PspC from the type 4 pneumococcus was obtained from The Institute for Genomic Research website at http://www.tigr.org.

Results

Binding of Complement Factor H by S. pneumoniae

Previous observations in our laboratory indicated that S. pneumoniae is capable of absorbing proteins from human plasma (unpublished results). To better investigate the nature of these interactions, a series of strains (Table 1), both encapsulated and unencapsulated, was incubated with plasma.

TABLE 1
Streptococcus pneumoniae strains

| Bacterial | | |
|---|---|---|
| Strains | Relevant Properties | Source of Reference |
| A66 | Capsulated clinical strain (type 3) | (38, 39) |
| PR218 | Km^{R}. Unencapsulated derivative of | Iannelli, Pearce, |
| | A66 | and Pozzi |
| | | (unpublished) |
| FP13 | Cm^{R}, Km^{R}, Hic, Mutant of PR218 | This work |
| | in which hic is deleted and | |
| | substituted with a cat cassette. | |
| D39 | Capsulated clinical strain (type 2) | (38, 40) |
| Rx1 | Unencapsulated derivative of D39 | (41) |
| PR201 | Km^{R}. Unencapsulated derivative of | Iannelli, Pearce, |
| | D39 | and Pozzi |
| | | (unpublished) |
| FP7 | PspC deletion mutant of Rx1 (in- | This work |
| | frame deletion of nucleotides 124 to | |
| | 1338, GenBank accession no. | |
| | AF067128) | |
| G54 | Capsulated clinical strain (type 19f) | (39) |
| PR212 | Km^{R}. Unencapsulated derivative of | Iannelli, Pearce, |
| | G54 | and Pozzi |
| | | (unpublished) |
| 3496 | Capsulated (type 3) | (39) |
| PR215 | Km^{R}. Unencapsulated derivative of | Iannelli, Pearce, |
| | 3496 | and Pozzi |
| | | (unpublished) |

After washing, plasma proteins bound to the pneumococci were eluted and separated by polyacrylamide gel electrophoresis (PAGE). In particular SDS-PAGE analysis of plasma proteins absorbed by and eluted from D39, HB565, 3496, G54, PR201, PR212, PR-18 and FP13 was carried out, Diluted plasma and pH were also ran on the gel. Strains D39 (type 2, encapsulated), and all four unencapsulated strains (serotypes 2, 3, 3, and 19) absorbed a protein with an estimated molecular mass of 140 kDa. In repeated experiments-PR218, an unencapsulated derivative (Pearce, Iannelli and Pozzi, manuscript in preparation) of Avery strain A66, consistently showed the most prominent absorption of the 140 kDa protein. The protein absorbed by PR218 was subjected to trypsin-in-gel digestion, and six internal framents were sequenced by Edman degradation.

These sequences showed 100% identity to various regions in human complement factor H. A replica of the gel obtained was transferred to a PVDF membrane by electroblotting, and probed with a rabbit anti-fH antiserum. The antiserum reacted with the aforementioned 140 kDa band, a band of similar size in plasma, and purified fH. There was also a weak reactivity with a band corresponding to appr. 50 kDa. When subjected to NH₂-terminal sequencing, the band was identified as human iminunoglobulin heavy chains, suggesting specific or unspecific binding of immunoglobulins to pneumococci, and subsequent crossreactivity with the secondary goat anti-rabbit antibodies.

To exclude that binding of fH was secondary to complement activation and deposition of C3 at the bacterial surface, the plasma absorption with strain D39 was repeated using a C3-deficient serum. The protein reacting with anti-f antibodies was present in equal amounts when comparing results from absorptions of normal human plasma and the C3-deficient serum (data not shown), showing that fH binding is independent of C3.

A group of S. pneumoniae strains was examined for binding of radiolabelled fH. Most strains showed significant binding of fH, although the degree of binding varied considerably between strains (Table 2).

TABLE 2
Binding of radiolabelled fH to pneumococci

FH binding (5)*

| Strain | type | encaps. | unencaps. |
|---|---|---|---|
| D39/PR201 | 2 | 2.3 | 3.6 |
| 3496/PR215 | 3 | 15.4 | 26.6 |
| HB565/PR218 | 3 | 27.6 | 40.8 |
| G54/PR212 | 19 | 33.9 | 35.9 |

*Four pairs of encapsulated/unencapsulated strains were examined. Binding is expressed as the percentage of added radioactivity. Results are means of two experiments with duplicate samples.

Encapsulated strains generally bound somewhat less than the corresponding unencapsulated strains. To investigate whether the binding was mediated by proteinaceous structures, binding of fH to strain PR218 was examined following treatment of bacteria with different proteases (data not shown). Binding was almost completely abolished by pretreatment with papain. Trypsin also caused a major decrease, whereas pepsin had a moderate effect (<50% decrease). These results confirm a previous observation (15) that fH binding can be abolished by pretreatment of bacteria with trypsin.

Identification and Sequence Analysis of a Candidate Gene Encoding a fH-Binding Protein

A recent paper (15) suggests that type 3 pneumococci are resistant to complement activation and phagocytosis by virtue of fH binding to proteinaceous surface structures. Previously described bacterial surface proteins known to interact with ft include streptococcal M proteins (11) and YadA (13) from Y. enterocolitica. Nucleotide and amino acid sequences of YadA, M1, M1.1 (26), M6 (27), and the M-like protein H (28), were used to search a pneumococcal (type 4 strain JNR. 7/87) genome database obtained from The Institute for Genomic Research (http://www.tigr.org). The highest scoring homology for all the probes was found in a 2106 bp open reading frame (ORF), encoding a putative protein of 702 amino acids (aa). The degree of homology between the pneumococcal protein and the fH-binding proteins used for searching was low, but significant, when comparing the full sequences. However, there were several limited regions of markedly higher homology. A GENBANK search with this putative protein identified it as an allele of PspC, also denoted SpsA, CbpA, or PbcA (16). PspC was then conversely used to search the Streptococcus pyogenes genome sequencing project, and the highest scoring match was found to encode the M1 protein.

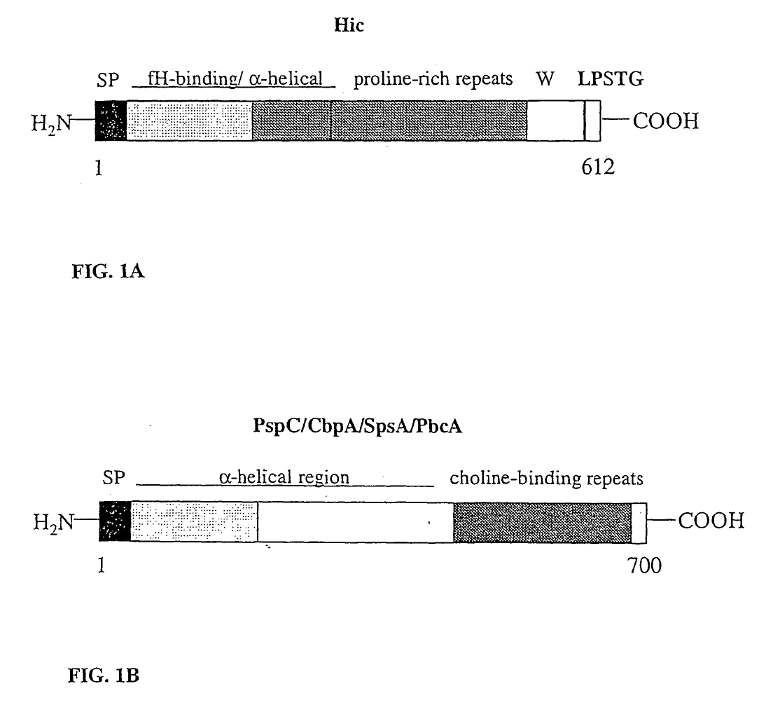

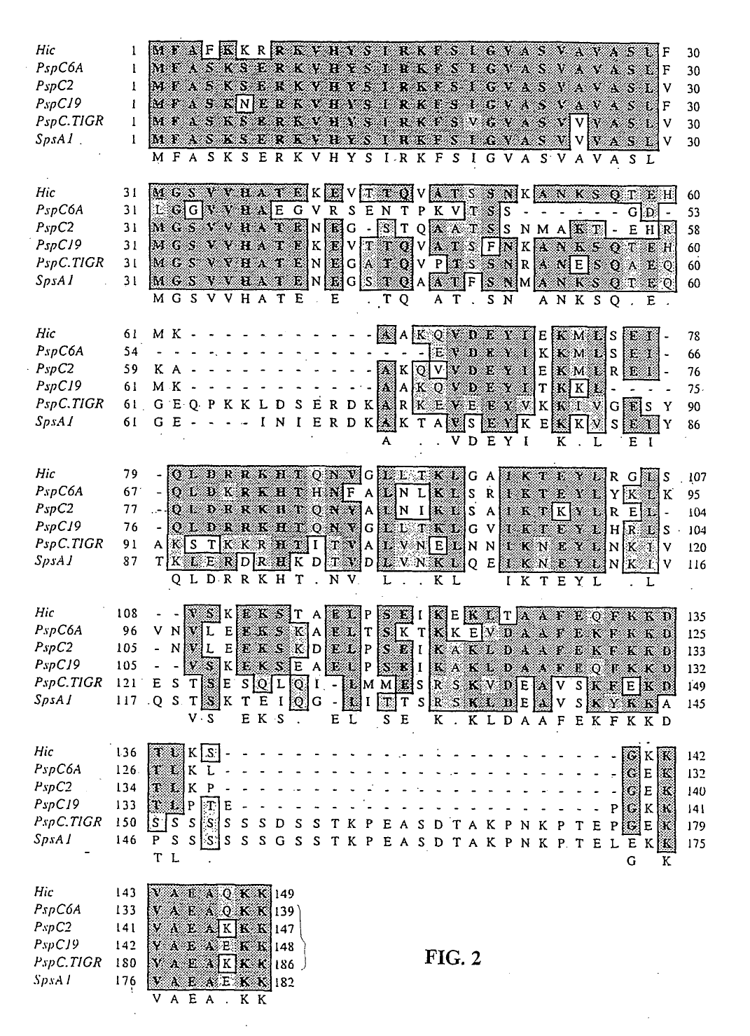

We chose to sequence the chromosomal locus of the pspC gene in Avery strain A66 (type 3) and its derivative PR218. Serotype 3 strains strongly resist phagocytosis (29), and PR218 shows a prominent absorption of fH from human plasma. The locus contained a 1836 bp ORF, encoding a putative protein of 612 aa. The gene was tentatively named hic, for factor H binding inhibitor of complement (GenBank accession number AF252857). The Hic protein (schematically depicted in FIG. 1, top) contains a proline-rich region consisting of 22 repeats of 11 amino acids. Near the COOH-terminus there is a consensus sequence LPSTGS, typical of Gram-positive cell wall-anchored proteins (30). This sequence is followed by a hydrophobic COOH-terminal tail. The Hic sequence was used to search the streptococcal Genome Project database, and identified M protein as the best match. Several pneumococcal surface proteins were found to be homologous to Hic. SpsA from type 2 and type 47 pneumococci contains a region highly homologous to Hic. SpsA binds secretory IgA and its secretory component (17). CbpA (1S), an adhesin and virulence determinant in type 2 pneumococci, also contains this region, as does PbcA (unpublished sequence from GENBANK). SpsA2, CbpA, and PbcA together form the D39-lineage of PspC alleles (16). The 149 aa long NH2-terminal region of Hic, including the predicted 37 aa leader peptide, was aligned with the corresponding region in PspC proteins from serotypes 1, 2, 4, 6A, and 19 (FIG. 2). No particular function has previously been suggested for this region in the PspC proteins. Interestingly, the remainder of Hic showed no significant homology to the PspC proteins except for shorter stretches in the proline-rich region of PspC. In contrast to Hic (FIG. 1, bottom), the PspC proteins contain a series of repeats with choline-binding motifs, a different mechanism for surface attachment. Computer predictions (31,32) of the secondary structure resulted in strong predictions of α-helical structure in the NH₂-terminal region of both Hic (aa 40-270) and PspC (aa 50-250).

Construction and Properties of a Hic-Deficient Mutant Strain

To investigate the possible contribution of Hic to pneumococcal fH-binding, the unencapsulated strain PR218 (serotype 3), showing the most pronounced absorption of fH from human plasma, was chosen for further studies. Splicing by overlap extension (gene SOEing) (19) was used to flank an antibioticum resistance cassette with sequences found up- and downstream of the hic gene. PR218 was transformed with this construct and the hic gene was deleted by double cross-over mutagenesis. The resulting strain, FP13, grew as well as the parent strain in ordinary growth media. The deletion of hic was confirmed by PCR experiments.

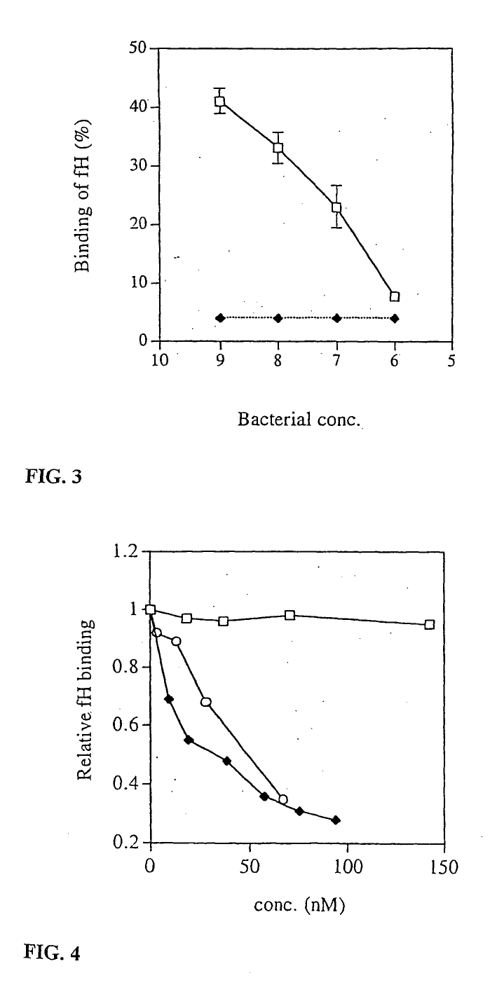

The mutant strain was subjected to plasma absorption experiments identical to those described above. In contrast to the parent strain, no band at the position of fH was detected with the anti-fH antiserum. Furthermore, the binding of radiolabelled fH to wild type and mutant bacteria was examined using serial dilutions of bacteria (FIG. 3). Unlike the parent strain PR218, the mutant strain FP 13 showed background levels of if binding even at the highest bacterial concentration. The possible contribution of PspC to pneumococcal fH binding was also examined by constructing a mutant derivative of D39, where the pspc gene has been truncated, so that the 405 NH2-terminal amino acids are missing in PspC. This mutant, called FP7, was used in plasma absorption experiments, and no band reacting with the anti-fH antibodies was eluted (data not shown).

Mapping of the Factor H-Binding Region of Hic

We decided to investigate the binding properties of the non-repeat region of Hic, including the part shared by Hic, PspC, CbpA, SpsA, and PbcA. Therefore, the hic region encoding the NH₂-terminal part of Hic (aa 39-261) was cloned into the vector pGEX, resulting in a fusion with the gene encoding GST. Control sequencing of the insert verified the presence of the partial hic gene, showing 100% identity with the DNA sequence from strain A66. GST:Hic³⁹⁻²⁶¹ and GST were overexpressed, affinity purified, and analysed by SDS-PAGE. Although partly degraded, the main protein band had the expected mass (54 kDa). An identical gel was blotted to a membrane, which was then incubated with radiolabelled fH, washed, and subjected to autoradiography. fH bound to GST:Hic³⁹⁻²⁶¹, but not to GST. The fusion protein and the GST control were also applied in serial dilutions (5, 1, 0.2 and 0.04 μg) onto a nitrocellulose membrane. The membrane was probed with radiolabelled fH, which showed binding to the fusion protein, and no binding to GST. Furthermore, the fusion protein was used in a competitive binding assay to investigate whether GST:Hic³⁹⁻²⁶¹ could compete with fH binding to PR218 bacteria. The result (FIG. 4) shows that the Hic domain of the fusion protein blocks bacterial fH binding, whereas GST alone does not affect binding. Similar experiments were performed with S. pneumoniae strains 3496, G54, PR215, and HB565. Also in these experiments GST:Hic 39-261 blocked the binding of fH to the tested strains, whereas GST had no effect (data not shown). The G54 strain is of serotype 19, and the similarity between Hic and PspC of a type 19 pneumococcus (see FIG. 1B) implicates that fH binding to non type 3 strains is mediated by the PspC region homologous to Hic.

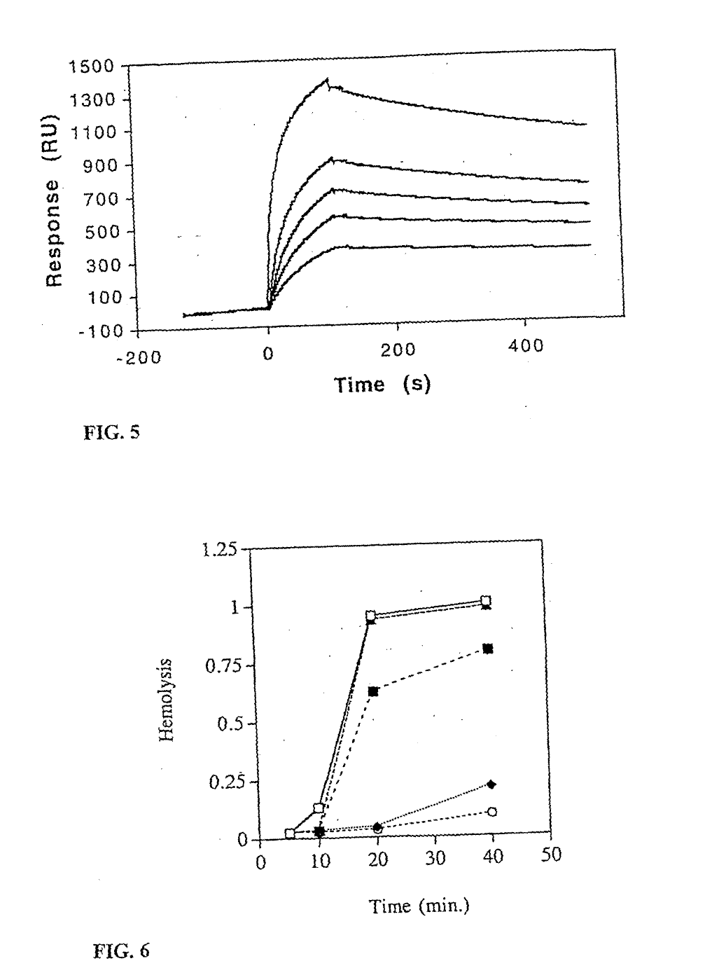

The interaction between GST:Hic³⁹⁻²⁶¹ and fH was further examined by surface plasmon resonance. Anti-GST antibodies were coupled to a carboxymethyldextrane chip, followed by the immobilization of GST:Hic³⁹⁻²⁶¹ as the ligand. Highly purified fH was used as the analyte at various concentrations. fH interacted with GST:Hic³⁹⁻²⁶¹ over the whole range of concentrations (0.1-2 μM), showing partial saturation at the highest fH concentration. The experiment was repeated three times, with independent couplings onto the chips. A representative experiment with consecutive regenerations of one chip is shown (FIG. 5). As a control, GST was similarity immobilized on an anti-GST antibodies chip. No binding of fluid phase fH was obtained (data not shown). A global analysis of data was performed to determine rate constants of association/dissociation and constants of association and dissociation, applying the standard Langmuir 1:1 model. Values are mean±SD from three different experiments.

| | kₐ | k_{d} | K_{A} | K_{D} |
|---|---|---|---|---|
| | (10⁴ × Ms⁻¹) | (10⁻⁴ × s⁻¹) | (10⁷ × M⁻¹) | (10⁻⁸ × M) |
| Hic + fH | 2.8 ± 0.24 | 6.1 ± 2.4 | 5.0 ± 1.9 | 2.3 ± 1.1 |

Hic and Complement Inhibition

From a functional point of view, an important question is whether the binding of Hic to fH affects the complement inhibitory function of fH. To address this issue we adapted a previously described methodology where complement-mediated lysis of rabbit erythrocytes in serum is inhibited by the addition of fH (24). By using a C2-deficient serum as a source of complement, any influence from the classical pathway was excluded. Initial experiments were performed and showed a dose-dependent inhibition of alternative-pathway mediated hemolysis when fH was added (data not shown). By increasing the concentration of fH in the reaction threefold (relative to fH in serum), a complete inhibition of hemolysis was achieved. Smaller fH increments resulted in partial inhibition of hemolysis. The effect of GST:Hic³⁹⁻²⁶¹, GST, or fH and GST:Hic³⁹⁻²⁶¹ (1:2 molar ratio) on hemolysis was investigated. A preincubation step (5 min) allowed some time for equilibration of the fH and GST:Hic³⁹⁻²⁶¹ interaction. A kinetic study of hemolysis showed that the presence of GST:Hic³⁹⁻²⁶¹ in the reaction did not decrease the fH-mediated inhibition of complement activation. Rather, the simultaneous presence of fH and GST:Hic³⁹⁻²⁶¹ resulted in increased inhibition. Interestingly, GST:Hic³¹⁻²⁶¹ seems to have an intrinsic complement inhibitory effect, as hemolysis was partially inhibited when the fusion protein alone (with no surplus fH) was added (FIG. 6).

### REFERENCES

1. AlonsoDeVelasco, E., Verheul, A. F. M., Verhoef, J., and Snippe, H. (1995) Microbiol. Rev. 59(4), 591-603

2. Kelly, T., Dillard, J. P., and Yother, J. (1994) Infect. Immun. 62(5), 1813-1819

3. Berry, A. M., Yother, J., Briles, D. E., Hansman, D., and Paton, J. C. (1989) Infect. Immun. 57, 2037-2042

4. McDaniel, L. S., Yother, J., Vijayalcumar, M., McGarry, L., Guild, W. R., and Briles, D. E. (1987) J. Exp. Med. 165, 381-394

5. Berry, A. M., and Paton, J. C. (1996) Infect. Immun. 64(12), 5255-5262

6. Brown, E. J. (1985) Curr. Top. Microbiol. Immunol. 121, 159-187

7. Gordon, D. L., Rice, J., Finlay-Jones, J. J., McDonald, P. J., and Hostetter, M. K. (1988) J. Infect. Dis. 157, 697-704

8. Wurzner, R. (1999) Mol. Immunol. 36,249-260

9. Zipfel, P. F., Hellwage, J., Friese, M. A., Hegasy, G., Jokiranta, S. T., and Meri, S. (1999) Mol. Immunol. 36, 241-248

10. Morgan, B. P., and Harris, C. L. (1999) Complement regulatory proteins, Academic press, San Diego

11. Horstmann, R. D., Sievertsen, H. J., Knobloch, J., and Fishetti, V. A. (1988) Proc. Natl. Acad. Sci. USA 85, 1657-1661

12. Johnsson, E., Berggard, K., Kotarsky, H., Hellwage, J., Zipfel, P. F., Sjobring, U., and Lindahl, G. (1998) J. Immunol. 161, 4894-4901

13. China, B., Sory, M.-P., N'guyen, B. T., De Bruyere, M., and Cornelis, G. R. (1993) Infect. Immun. 61(8), 3129-3136

14. Tu, A.-H. T.,-Fulgham, R. L., McCrory, M. A., Briles, D. E., and Szalai, A. J. (1999) Infect. Immun. 67(9), 4720-4724

15. Neeleman, C., Geelen, S. P. M., Aerts, P. C., Daha, M. R., Mollnes, T. E., Roord, J. J., Posthuma, G., Van Dijk, H., and Fleer, A. (1999) infect. Immun. 67(9), 45174524

16. Brooks-Walter, A., Briles, D. E., and Hollingshead, S. K. (1999) Infect. Immun. 67(12), 6533-6542

17. Hammerschmidt, S., Talay, S. R., Brandtzaeg, P., and Chhatwal, G. S. (1997) Mol. Microbiol. 25(6), 1113-1124

18. Rosenow, C., Ryan, P., Weiser, J. N., Johnson, S., Fontan, P., Ortqvist, A., and Masure, R. (1997) Mol. Microbiol. 25(5), 819-829

19. Horton, R. M. (1995) Mol. Biotechnol. 3, 93-99

20. Laemmli, U. K. (1970) Nature 227, 680-685

21. Towbin, H., Staelielin, T., and Gordon, J. (1979) Proc. Natl. Acad. Sci. USA 76(9), 4350-4354

22. Nesbitt, S. A., and Horton, M. A. (1992) Anal. Biochem. 206, 267-272

23. Åkesson, P., Cooney, J., Kishimoto, F., and Björck, L. (1990) Mol. Immunol. 27(6), 523-531

24. Nydegger, U. E., Fearon, D. T., and Austen, K. F. (1978) J. Immunol. 120, 14041408

25. Truedsson, L., Alper, C. A., Awdeh, Z. L., Johansen, P., Sjöholm, A. G., and Sturfelt, G. (1993) J. Immunol. 151, 5856-5863

26. Harbaugh, M. P., Podbielski, A. P., Hugl, S., and Cleary, P. (1993) Mol. Microbiol. 8, 981-991

27. Hollingshead, S. K., Fischetti, V. A., and Scott, J. R. (1986) J. Biol. Chem. 261, 1677-1686

28. Gomi, HI., Hozuini, T., Hattori, S., Tagawa, C., Kishimoto, F., and Björck, L. (1990) J. Immunol. 144, 4046-4052

29. Hostetter, M. K. (1986) J. Infect. Dis. 153, 682-693

30. Navarre, W. W., and Schneewind, 0. (1999) Microbiol. Mol. Biol. Rev. 63(1), 174-229

31. Chou, P. Y., and Fasman, G. D. (1974) Adv. Enzymol. Relat. Areas Mol. Biol. 47, 45-148

32. Gamier, J., Osguthorpe, D. J., and Robson, B. (1978) J. Mol. Biol. 120, 97-120

33. Bruyn, G. A. W., Zegers, B. J. M., and van Furth, R. (1992) Clin. Infect. Dis. 14, 251-262

34. Johnsson, E., Thern, A., Dahlback, B., Heden, L.-O., Wikstrom, M., and Lindahl, G. (1996) J. Immunol. 157, 3021-3029

35. Angel, C. S., Ruzek, M., and Hostetter, M. K. (1994) J. Infect. Dis. 170, 600-608

36. Ram, S., Mcquillen, D. P., Gulati, S., Elkins, C., Pangburn, M. K., and Rice, P. A. (1998) J. Exp. Med. 188, 671-680

37. Ram, S., Sharma, A. K., Simpson, S. D., Gulati, S., Mcquillen, D. P., Pangburn, M. K., and Rice, P. A. (1998) J. Exp. Med. 187, 743-752

38. Avery, O. T., MacLeod, C. M., and McCarty, M. (1944) 3. Exp. Med. 79, 137-158

39. Pozzi, G., Masala, L., lannelli, F., Manganelli, R., Havarstein, L. S., Piccoli, L., Simon, D., and Morrison, D. A. (1996) J. Bacteriol. 178, 6087-6090

40. Iannelli, F., Pearce, B. J., and Pozzi, G. (1999) J. Bacteriol. 81, 2652-2654

41. Shoemaker, N. B., and Guild, W. R. (1974) Mol. Gen. Genet. 128, 283-290

1. A polypeptide having the ability to bind factor H comprising:
(a) the amino acid sequence of Thr38 to Lys149 of SEQ ID NO: 1
(b) a variant of (a) which is capable of binding factor H, or
(c) a fragment of (a) or (b) of at least 20 amino acids in length which is capable of binding factor H, for use in prophylaxis or therapy.

2. A vaccine composition comprising a polypeptide having an amino acid sequence selected from:
(a) the amino acid sequence of Thr38 to Lys149 of SEQ ID NO: 1,
(b) a variant of (a) which is capable of generating an immune response to Streptococcus pneumoniae or binding to an anti-protein Hic antibody, or
(c) a fragment of (a) or (b) of at least 6 amino acids in length which is capable of generating an immune response against Streptococcus pneumoniae, or of binding to an anti protein Hic antibody.

3. A polypeptide according to claim 1 or claim 2, wherein the variant (b) has at least 50%, preferably at least 60%, and most preferably at least 70% sequence identity with the sequence (a).

4. A polypeptide of 15 to 800 amino acids in length comprising:
(a) the amino acid sequence of SEQ ID NO: 1, or
(b) a polypeptide showing sequence identity of at least 85%, preferably at least 95%, and most preferably at least 99% sequence identity with (a).

5. A polynucleotide encoding a polypeptide as defined in any one of claims 1 to 4, the polynculeotide comprising:
(i) the nucleotide coding sequence of SEQ ID NO: 4 or a sequence complementary thereto,
(ii) a nucleotide sequence which selectively hybridizes to said sequence (i) or a fragment thereof, or
(iii) a nucleotide sequence which codes for a polypeptide having the same amino acid sequence as that encoded by a said sequence of (i) or (ii).

6. A factor H-binding protein comprising an amino-terminal amino acid sequence showing a homology of at least 50%, preferably at least 60%, and most preferably at least 70% with the subsequence from Thr38 to Lys149 of the sequence of SEQ ID NO: 1, for medical use.

7. A factor H-binding peptide comprising 20-200 amino acids, said peptide showing a homology of at least 50%, preferably at least 60%, and most preferably at least 70% with the subsequence from Thr38 to Lys149 of the sequence of SEQ ID NO: 1, for medical use.

8. A Hic protein-derived polypeptide comprising 15-800 amino acids which polypeptide shows a homology of at least 85%, preferably at least 95%, and most preferably at least 99% with the amino acid sequence of SEQ ID NO: 1.

9. A polypeptide according to claim 4 or 8, for medical use.

10. A nucleic acid sequence encoding a factor H-binding protein or peptide according to any one of claims 6 to 8.

11. A recombinant vector comprising a polynucleotide according to claim 5 or claim 10.

12. A vector according to claim 11 wherein the vector is an expression vector and said polynucleotide is operably linked to a regulatory sequence.

13. A host cell transformed with a vector according to claim 11 or claim 12.

14. An isolated antibody, preferably a monoclonal antibody, specifically binding a polypeptide according to any one of claims 1 to 4 or 6 to 8.

15. A pharmaceutical composition comprising a polypeptide according to any one of claims 1 to 4 or claims 6 to 8, together with a pharmaceutically acceptable carrier, excipient or diluent.

16. Use of a polypeptide according to anyone of claims 1 to 4 or claims 6 to 8 for preparing a vaccine composition against Streptococcus pneuminiae infections.

17. A method of identifying an agent which inhibits binding of factor H to Streptococcus pneuminiae comprises:
(a) providing a polypeptide as defined in claims 1 to 4 or 6 to 8,
(b) incubating said polypeptide with factor H and a test agent,
(c) monitoring for binding of factor H to the polypeptide of (a) and determining thereby whether the test agent inhibits binding of factor H to the polypeptide of (a).

18. A method of inhibiting the binding of factor H by Streptococcus pneumoniae comprising identifying a substance which inhibits the binding of factor H according to claim 17, and using an agent so identified to inhibit binding of factor H to Streptococcus pneumoniae.


SEQUENCE LISTING (11 sequences)

SEQ ID NO: 1 - protein, 612 aa, Streptococcus pneumoniae
MFAFKKRRKVHYSIRKFSIGVASVAVASLFMGSVVHATEKEVTTQVATSSNKANKSQTEHMKAAKQVDEYIEKMLSEIQLDRRKHTQNVGLLTKLGAIKTEYLRGLSVSKEKSTAELPSEIKEKLTAAFEQFKKDTLKSGKKVAEAQKKAKDQKEAKQEIEALIVKHKGREIDLDRKKAKAAVTEHLKKLLNDIEKNLKKEQHTHTVELIKNLKDIEKTYLHKLDESTQKAQLQKLIAESQSKLDEAFSKFKNGLSSSSNSGSSTKPETPQPETPKPEVKPELETPKPEVKPEPETPKPEVKPEPETPKPEVKPELETPKPEVKPEPETPKPEVKPEPETPKPEVKPEPETPKPEVKPELETPKPEVKPELETPKPEVKPEPETPKPEVKPELETPKPEVKPEPETPKPEVKPELETPKPEVKPEPETPKPEVKPELETPKPEVKPEPETPKPEVKPEPETPKPEVKPEPETPKPEVKPELETPKQKVKPEPETPKPEVKPEPETPKPEVKPELETPKPEVKPELEIPKPEVKPDNSKPQADDKKPSTPNNLSKDKQSSNQASTNENKKQGPATNKPKKSLPSTGSISNLALEIAGLLTLAGATILAKKRMK

SEQ ID NO: 2 - DNA, 27 bp, Streptococcus pneumoniae
tgggatcccagagaaggaggtaactac

SEQ ID NO: 3 - DNA, 20 bp, Streptococcus pneumoniae
ggagcctgaattcgacgaag

SEQ ID NO: 4 - DNA, 1839 bp, Streptococcus pneumoniae
atgtttgcattcaaaaaacgaagaaaagtacattattcaattcgtaaatttagtattggagtagctagtgtagctgttgccagtctttttatgggaagtgtggttcatgcgacagagaaggaggtaactacccaagtagccacttcttctaataaggcaaataaaagtcagacagaacatatgaaagctgctaaacaagtcgatgaatatatagaaaaaatgttgagtgagatccaattagatagaagaaaacatacccaaaatgtcggcttactcacaaagttgggcgcaattaaaacggagtatttgcgtggattaagtgtttcaaaagagaagtcgacagctgagttgccgtcagaaataaaagaaaagttaaccgcagcttttgagcagtttaaaaaagatacattgaaatcaggaaaaaaggtagcagaagctcagaaaaaagccaaggatcaaaaagaagctaaacaggagatagaagctctaatcgttaaacataaggggcgagaaatcgatttagatcgaaagaaggcaaaggctgcagttactgaacatctaaaaaaattattgaatgacatcgagaaaaatttaaaaaaagagcaacatacccatactgtagagttaattaaaaacttgaaagatattgaaaaaacgtatttgcataagttagatgaatcaacgcaaaaagcccaactacagaaactgatcgcagaaagtcaatcaaaactagatgaagctttttctaaatttaaaaatggcttatcttcttcgtcgaattcaggctcctccactaaaccagaaactccgcagccggaaacaccaaaaccagaggttaaaccagagctggaaacaccaaaaccagaggttaaaccagagccggaaacaccaaaaccagaggttaaaccagagccggaaacaccaaaaccagaggttaaaccagagctggaaacaccaaaaccagaggttaaaccagagccggaaacaccaaaaccagaggttaaaccagagccggaaacaccaaaaccagaggttaaaccagagccggaaacaccaaaaccagaggttaaaccagagctggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagccggaaacaccaaaaccagaggttaaaccagagccggaaacaccaaaaccagaggttaaaccagagctggaaacaccaaaacagaaggttaaaccagagccggaaacaccaaaaccagaggttaaaccagagccggaaacaccaaaaccagaggttaaaccagagctggaaacaccaaaaccagaggttaaaccagagctggaaataccaaaaccagaggttaaaccagataatagcaagccacaagcagatgataagaagccatcaactccaaataatttaagcaaggacaagcaatcttctaaccaagcttcaacaaacgaaaacaagaagcaaggtccagcaacaaataaaccgaagaagtcattgccatcaactggatctatttcaaatctagcacttgaaattgcaggtcttcttaccttggcgggggcaaccattcttgctaagaaaagaatgaaatag

SEQ ID NO: 5 - protein, 488 aa, Streptococcus pneumonie
MFASKSERKVHYSIRKFSIGVASVAVASLFLGGVVHAEGVRSENTPKVTSSGDEVDEYIKKMLSEIQLDKRKHTHNFALNLKLSRIKTEYLYKLKVNVLEEKSKAELTSKTKKEVDAAFEKFKKDTLKLGEKVAEAQKKVEEAKKKAKDQKEEDHRNYPTNTYKTLELEIAESDVKVKEAELELLKEEAKTRNEDTINQAKAKVKSEQAEATRLKKIKTDREQAEATRLENIKTDREKAEEAKRKAEAEEVKDKLKRRTKRAVPGEPATPDKKENDAKSSDSSVGEETLPSPSLKSGKKVAEAQKKVAEAEKKAKDQKEEDRRNYPTNTYKTLDLEIAESDVKVKEAELELVKEEAKESRNEEKVKQAKAKVESKKAEATRLEKIKTDRKKAEEAKRRAAEEDKVKEKPAEQPQPAPAPQPEKPTEEPENPAPAPKPENPAEQPKAEKPADQQAEEDYARRSEEEYNRLTQQQPPKTEKPAQPSTPKT

SEQ ID NO: 6 - DNA, 1464 bp, Streptococcus pneumoniae
atgtttgcatcyaaaagcgaaagaaaagtacattattcaattcgtaaatttagtattggagtagctagtgtagctgttgctagcttgttcttaggaggagtagtccatgcagaaggggttagaagtgagaatacccccaaggttacatctagtggggatgaagtcgatgaatatataaaaaaaatgttgagtgagatccaattagataaaagaaaacatacccacaatttcgccttaaacctaaagttgagcagaattaaaacggagtatttgtataaattaaaagttaatgttttagaagaaaagtcaaaagctgagttgacgtcaaaaacaaaaaaagaggtagacgcagcttttgagaagtttaaaaaagatacattgaaactaggagaaaaggtagcagaagctcagaagaaggttgaagaagctaagaaaaaagccaaggatcaaaaagaagaagatcaccgtaactacccaaccaatacttacaaaacgcttgaacttgaaattgctgagtccgatgtgaaagttaaagaagcggagcttgaactattgaaagaggaagctaaaactcgaaacgaggacacaattaaccaagcaaaagcgaaagttaagagtgaacaagctgaggctacaaggttaaaaaaaatcaagacagatcgtgaacaagctgaggctacaaggttagaaaacatcaagacagatcgtgaaaaagcagaagaagctaaacgaaaagcagaagcagaagaagttaaagataaactaaagaggcggacaaaacgagcagttcctggagagccagcaacacctgataaaaaagaaaatgatgcgaagtcttcagattctagcgtaggtgaagaaactcttccaagcccatccctgaaatcaggaaaaaaggtagcagaagctcagaagaaggtagcagaagctgagaaaaaagccaaggatcaaaaagaagaagatcgccgtaactacccaaccaatacttacaaaacgcttgaccttgaaattgctgagtccgatgtgaaagttaaagaagcggagcttgaactagtaaaagaggaagctaaggaatctcgaaacgaggaaaaagttaagcaagcaaaagcgaaagttgagagtaaaaaagctgaggctacaaggttagaaaaaatcaagacagatcgtaaaaaagcagaagaagctaaacgaagagcagcagaagaagataaagttaaagaaaaaccagctgaacaaccacaaccagcgccggctcctcaaccagaaaaaccaactgaagagcctgagaatccagctccagctccaaaacctgagaatccagctgaacaaccaaaagcagaaaaaccagctgatcaacaagctgaagaagactatgctcgtagatcagaagaagaatataatcgcttgactcaacagcaaccgccaaaaactgaaaaaccagcacaaccatctactccaaaaaca

SEQ ID NO: 7 - protein, 701 aa, Streptococcus pneumoniae
  PEPTIDE (650)...(650): Xaa = Asn, Asp, His or Tyr
MFASKSERKVHYSIRKFSIGVASVAVASLVMGSVVHATENEGSTQAATSSNMAKTEHRKAAKQVVDEYIEKMLREIQLDRRKHTQNVALNIKLSAIKTKYLRELNVLEEKSKDELPSEIKAKLDAAFEKFKKDTLKPGEKVAEAKKKVEEAKKKAEDQKEEDRRNYPTNTYKTLELEIAEFDVKVKEAELELVKEEAKESRNEGTIKQAKEKVESKKAEATRLENIKTDRKKAEEEAKRKADGKLKEANVATSDQGKPKGRAKRGVPGELATPDKKENDAKSSDSSVGEETLPSSSLKSGKKVAEAEKKVEEAEKKAKDQKEEDRRNYPTNTYKTLDLEIAESDVKVKEAELELVKEEAKEPRDEEKIKQAKAKVESKKAEATRLENIKTDRKKAEEEAKRKAAEEDKVKEKPAEQPQPAPATQPEKPAPKPEKPAEQPKAEKTDDQQAEEDYARRSEEEYNRLTQQQPPKTEKPAQPSTPKTGWKQENGMWYFYNTDGSMATGWLQNNGSWYYLNANGAMATGWLQNNGSWYYLNANGSMATGWLQNNGSWYYLNANGAMATGWLQYNGSWYYLNSNGAMATGWLQYNGSWYYLNANGDMATGWLQNNGSWYYLNANGDMATGWLQYNGSWYYLNANGDMATGWVKDGXTWYYLKASGAMKASQWFKVSDKWYYVNGSGALAVNTTVDGYGVNANGEWVN

SEQ ID NO: 8 - DNA, 2106 bp, Streptococcus pneumoniae
  misc_feature (1948)...(1948): n equals any nucleotide
atgtttgcatcaaaaagcgaaagaaaagtacattattcaattcgtaaatttagtattggagtagctagtgtagctgttgccagtcttgttatgggaagtgtggttcatgcgacagagaacgagggaagtacccaagcagccacttcttctaatatggcaaagacagaacataggaaagctgctaaacaagtcgtcgatgaatatatagaaaaaatgttgagggagattcaactagatagaagaaaacatacccaaaatgtcgccttaaacataaagttgagcgcaattaaaacgaagtatttgcgtgaattaaatgttttagaagagaagtcgaaagatgagttgccgtcagaaataaaagcaaagttagacgcagcttttgagaagtttaaaaaagatacattgaaaccaggagaaaaggtagcagaagctaagaagaaggttgaagaagctaagaaaaaagccgaggatcaaaaagaagaagatcgtcgtaactacccaaccaatacttacaaaacgcttgaacttgaaattgctgagttcgatgtgaaagttaaagaagcggagcttgaactagtaaaagaggaagctaaagaatctcgaaacgagggcacaattaagcaagcaaaagagaaagttgagagtaaaaaagctgaggctacaaggttagaaaacatcaagacagatcgtaaaaaagcagaagaagaagctaaacgaaaagcagatggtaagttgaaggaagctaatgtagcgacttcagatcaaggtaaaccaaaggggcgggcaaaacgaggagttcctggagagctagcaacacctgataaaaaagaaaatgatgcgaagtcttcagattctagcgtaggtgaagaaactcttccaagctcatccctgaaatcaggaaaaaaggtagcagaagctgagaagaaggttgaagaagctgagaaaaaagccaaggatcaaaaagaagaagatcgccgtaactacccaaccaatacttacaaaacgcttgaccttgaaattgctgagtccgatgtgaaagttaaagaagcggagcttgaactagtaaaagaggaagctaaggaacctcgagacgaggaaaaaattaagcaagcaaaagcgaaagttgagagtaaaaaagctgaggctacaaggttagaaaacatcaagacagatcgtaaaaaagcagaagaagaagctaaacgaaaagcagcagaagaagataaagttaaagaaaaaccagctgaacaaccacaaccagcgccggctactcaaccagaaaaaccagctccaaaaccagagaagccagctgaacaaccaaaagcagaaaaaacagatgatcaacaagctgaagaagactatgctcgtagatcagaagaagaatataatcgcttgactcaacagcaaccgccaaaaactgaaaaaccagcacaaccatctactccaaaaacaggctggaaacaagaaaacggtatgtggtacttctacaatactgatggttcaatggcaacaggatggctccaaaacaacggttcatggtactatctaaacgctaatggtgctatggcgacaggatggctccaaaacaatggttcatggtactatctaaacgctaatggttcaatggcaacaggatggctccaaaacaatggttcatggtactacctaaacgctaatggtgctatggcgacaggatggctccaatacaatggttcatggtactacctaaacagcaatggcgctatggcgacaggatggctccaatacaatggctcatggtactacctcaacgctaatggtgatatggcgacaggatggctccaaaacaacggttcatggtactacctcaacgctaatggtgatatggcgacaggatggctccaatacaacggttcatggtattacctcaacgctaatggtgatatggcgacaggttgggtgaaagatgganatacctggtactatcttaaagcatcaggtgctatgaaagcaagccaatggttcaaagtatcagataaatggtactatgtcaatggctcaggtgcccttgcagtcaacacaactgtagatggctatggagtcaatgccaatggtgaatgggtaaactaa

SEQ ID NO: 9 - protein, 487 aa, Streptococcus pneumoniae
MFASKNERKVHYSIRKFSIGVASVAVASLFMGSVVHATEKEVTTQVATSFNKANKSQTEHMKAAKQVDEYITKKLQLDRRKHTQNVGLLTKLGVIKTEYLHRLSVSKEKSEAELPSEIKAKLDAAFEQFKKDTLPTEPGKKVAEAEKKVEEAKKKAEDQKEEDRRNYPTITYKTLELEIAESDVEVKKAELELVKEEAKGSRNEQKVNQAKAKVESKQAEATRLKKIKTDREQAETTRLENIKTDREKAEEAKRKADAKEQDESKRRVKGGVPGEQATLDKKENDAKSSDSSVGEETLPSPSLKSGKKVAEAEKKVAEAEKKAKDQKEEDRRNYPTNTYKTLELEIAESDVKVKEAELELVKEEAKESRNEEKVKQAKAEVESKKAEATRLEKIKTDRKKAEEAKRKAAEEDKVKEKPAEQPQPAPAPQPEKPAPAPKPENPAEQPKAEKPADQQAEEDYARRSEEEYNRLTQQQPPKTEKPAQPST

SEQ ID NO: 10 - protein, 693 aa, Streptococcus pneumoniae
MFASKSERKVHYSIRKFSVGVASVVVASLVMGSVVHATENEGATQVPTSSNRANESQAEQGEQPKKLDSERDKARKEVEEYVKKIVGESYAKSTKKRHTITVALVNELNNIKNEYLNKIVESTSESQLQILMMESRSKVDEAVSKFEKDSSSSSSSDSSTKPEASDTAKPNKPTEPGEKVAEAKKKVEEAEKKAKDQKEEDRRNYPTITYKTLELEIAESDVEVKKAELELVKVKANEPRDEQKIKQAEAEVESKQAEATRLKKIKTDREEAEEEAKRRADAKEQGKPKGRAKRGVPGELATPDKKENDAKSSDSSVGEETLPSPSLKPEKKVAEAEKKVEEAKKKAEDQKEEDRRNYPTNTYKTLELEIAESDVEVKKAELELVKEEAKEPRNEEKVKQAKAEVESKKAEATRLEKIKTDRKKAEEEAKRKAAEEDKVKEKPAEQPQPAPAPKAEKPAPAPKPENPAEQPKAEKPADQQAEEDYARRSEEEYNRLTQQQPPKTEKPAQPSTPKTGWKQENGMWYFYNTDGSMATGWLQNNGSWYYLNSNGAMATGWLQNNGSWYYLNANGSMATGWLQNNGSWYYLNANGSMATGWLQYNGSWYYLNANGSMATGWLQYNGSWYYLNANGDMATGWVKDGDTWYYLEASGAMKASQWFKVSDKWYYVNGSGALAVNTTVDGYGVNANGEWVN

SEQ ID NO: 11 - protein, 523 aa, Streptococcus pneumoniae
MFASKSERKVHYSIRKFSIGVASVVVASLVMGSVVHATENEGSTQAATFSNMANKSQTEQGEINIERDKAKTAVSEYKEKKVSEIYTKLERDRHKDTVDLVNKLQEIKNEYLNKIVQSTSKTEIQGLITTSRSKLDEAVSKYKKAPSSSSSSGSSTKPEASDTAKPNKPTELEKKVAEAEKKVEEAKKKAKDQKEEDYRNYPTITYKTLELEIAESDVEVKKAELELVKEEAKEPRNEEKVKQAKAKVESEETEATRLEKIKTDRKKAEEEAKRKAAEEDKVKEKPAEQQAEEDYARRSEEEYNRLTQQQPPKTEKPAQPSTPKTGWKQENGMWYFYNTDGSMATGWLQNNGSWYYLNSNGAMATGWLQNNGSWYYLNANGSMATGWLQNNGSWYYLNANGSMATGWLQYNGSWYYLNANGDMATGWLQYNGSWYYLNANGDMATGWLQYNGSWYYLNANGDMATGWVKDGDTWYYLEASGAMKASQWFKVSDKWYYVNGSGALAVNTTVDGYGVNANGEWVN